(19) United States
(12) Patent Application Publication
(10) Pub. No.: US 2019/0091076 A1
(43) Pub. Date: Mar. 28, 2019

(54) ABSORBENT ARTICLE WITH CHANNEL

(71) Applicant: Kimberly-Clark Worldwide Inc., Neenah, WI (US)

(72) Inventors: Yishal Y. Joshi, Medellin (CO); SangWook Lee, Medellin (CO); Shruti Aryal, Medellin (CO); Drea C. Bruss, Oshkosh, WI (US); John J. Garcia, Neenah, WI (US); Jose A. Peredo, Envigado (CO)

(21) Appl. No.: 16/078,142

(22) PCT Filed: Feb. 29, 2016
(86) PCT No.: PCT/US16/20062

§ 371 Date: Aug. 21, 2018

(51) Int. Cl. A61F 13/537; A61F 13/15

(52) U.S. Cl. CPC A61F 13/53713; A61F 13/15203; A61F 2013/53445; A61F 2013/53765; A61F 2013/15422

(57) ABSTRACT

An absorbent article which can have an improved body exudate intake capability. The absorbent article can have a topsheet layer, a backsheet layer, and an absorbent system positioned between the topsheet layer and the backsheet layer. The absorbent system can have at least a fluid intake layer and an absorbent core. The fluid intake layer and the absorbent core can together define an aligned channel in the absorbent system. The aligned channel can create a void space in the depth direction of the absorbent article wherein at least a portion of the topsheet layer can be maintained in a spaced apart relationship from the bottom of the aligned channel.

### BACKGROUND OF THE DISCLOSURE

A primary function of a personal care absorbent article is to retain and absorb body exudates. A desired attribute of the personal care absorbent article is to minimize the leakage of such body exudates from the absorbent article. It is also desired that the personal care absorbent article retain and absorb the body exudates in such a fashion so as to provide a dry feel to the wearer, removing body exudates from the skin at the time of the initial insult of the body exudate as well as retaining the body exudates in a location away from the skin after such insult.

Personal care absorbent articles, however, traditionally fail to possess the combination of the desired attributes. Absorbent articles commonly fail before the total absorbent capacity of the absorbent article is utilized. Problems which can typically exist can relate to the ability of the absorbent article to allow quick intake of the body exudate in one direction away from the topsheet layer and towards an absorbent core while preventing return of the body exudate in the opposite direction away from the absorbent core and toward the topsheet layer.

An additional issue may exist with personal care absorbent articles when attempting to retain and absorb body exudates having a higher viscosity that proves difficult to absorb and retain. Such body exudates may have difficulty penetrating the topsheet layer of the absorbent article as easily as a low viscosity body exudate, such as urine, and may spread across the surface of the topsheet layer of the absorbent article. These body exudates can move around on the topsheet layer of the absorbent article under an influence of gravity, motion, and pressure by the wearer of the absorbent article. The migration of the body exudates is often towards the perimeter of the absorbent article, increasing the likelihood of leakage from the absorbent article and smears against the skin of the wearer which can make clean-up of the skin difficult.

There remains a need for an absorbent article that can adequately reduce the incidence of leakage of body exudates from the absorbent article. There remains a need for an absorbent article which can provide improved handling of body exudates. There remains a need for an absorbent article that can minimize the amount of body exudates in contact with the wearer's skin.

### SUMMARY OF THE DISCLOSURE

An absorbent article characterized by comprising a longitudinal direction, a transverse direction, and a depth direction; a topsheet layer and a backsheet layer; and an absorbent system positioned between the topsheet layer and the backsheet layer, the absorbent system comprising a fluid intake layer positioned below the topsheet layer in the depth direction of the absorbent article, wherein the fluid intake layer comprises a fluid intake layer channel; an absorbent core positioned below the fluid intake layer in the depth direction of the absorbent article, wherein the absorbent core comprises a fluid intake layer; wherein the fluid intake layer channel is in a nested configuration with the absorbent core channel to form an aligned channel of the absorbent article, the aligned channel comprising an aligned channel bottom surface and an aligned channel perimeter side wall; and wherein the topsheet layer is in a spaced apart relationship from the aligned channel bottom surface resulting in a spatial gap between the topsheet layer and the bottom surface of the aligned channel.

In various embodiments, the aligned channel is elongate. In various embodiments, the aligned channel is oriented in the longitudinal direction of the absorbent article. In various embodiments, the aligned channel comprises a shape wherein the shape is one of ovular, circular, rectangular, square or triangular.

In various embodiments, a first portion of the absorbent system has a first height and a second portion of the absorbent system has a second height and the first height is different from the second height.

In various embodiments, a first portion of the absorbent system has a first density and a second portion of the absorbent system has a second density and the first density is different from the second density. In various embodiments, the first density is greater than the second density. In various embodiments, the first density is less than the second density.

In various embodiments, the topsheet layer comprises a surfactant.

In various embodiments, the fluid intake layer comprises a surfactant.

In various embodiments, the absorbent core comprises at least two separate layers.

In various embodiments, the aligned channel has a longitudinal length from about 15 mm to about 150 mm. In various embodiments, the aligned channel has a transverse width from about 10 mm to about 90 mm. In various embodiments, the height of the spatial gap is from about 1 mm to about 8 mm.

In various embodiments, the fluid intake layer is formed of a moisture insensitive material and the absorbent core is formed of a moisture sensitive material.

### BRIEF DESCRIPTION OF DRAWINGS

FIG. 1 is an illustration of a perspective view of an exemplary embodiment of an absorbent article.

FIG. 2 is an illustration of an exploded cross-sectional view of the absorbent article of FIG. 1 taken along line 2-2.

FIG. 3 is an illustration of a cross-sectional view of the absorbent article of FIG. 1 taken along line 2-2.

FIG. 4 is an illustration of an exploded cross-sectional view of an exemplary embodiment of an absorbent article having a multi-layer absorbent core.

FIG. 5 is an illustration of a cross-sectional view of an exemplary embodiment of an absorbent article having a multi-layer absorbent core.

Repeat use of reference characters in the present specification and drawings is intended to represent the same or analogous features or elements of the disclosure.

### DETAILED DESCRIPTION OF THE DISCLOSURE

The present disclosure is generally directed towards an absorbent article which can have an improved body exudate intake capability. An absorbent article can have a longitudinal direction, a transverse direction, and a depth direction. The absorbent article can have a topsheet layer, a backsheet layer, and an absorbent system positioned between the topsheet layer and the backsheet layer. The absorbent system can have at least a fluid intake layer and an absorbent core. The fluid intake layer can be positioned between the topsheet layer and the absorbent core in the depth direction of the absorbent article. The fluid intake layer and the absorbent core can together define an aligned channel in the absorbent system. The aligned channel can create a void space in the depth direction of the absorbent article wherein at least a portion of the topsheet layer can be maintained in a spaced apart relationship from the bottom of the aligned channel. In various embodiments, the topsheet layer and/or the fluid intake layer can be treated with a surfactant.

Definitions:

The term “absorbent article” refers herein to a garment or other end-use personal care absorbent article which may be placed against or in proximity to the body (i.e., contiguous with the body) of the wearer to absorb and contain various liquid, solid, and semi-solid body exudates discharged from the body. Such absorbent articles, as described herein, are intended to be discarded after a limited period of use instead of being laundered or otherwise restored for use. Such absorbent articles include, but are not limited to, diapers, diaper pants, training pants, youth pants, swim pants, feminine hygiene products, including, but not limited to, sanitary napkins, feminine pads, pantiliners, and panty shields, incontinence products, medical garments, surgical pads, and bandages, other personal care or health care garments, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber. Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

The term “thermoplastic” refers herein to a material which softens and which can be shaped when exposed to heat and which substantially returns to a non-softened condition when cooled.

The term “wearer” refers herein to one who uses an absorbent article, such as, but not limited to, a diaper, training pant, youth pant, incontinent product, feminine napkin, or other absorbent article and the absorbent article is placed in proximity to the body to capture body exudates.

Absorbent Article:

The present disclosure is generally directed towards an absorbent article which can have an improved body exudate intake capability. An absorbent article can have a longitudinal direction, a transverse direction, and a depth direction. The absorbent article can have a topsheet layer, a backsheet layer, and an absorbent system positioned between the topsheet layer and the backsheet layer. The absorbent system can have at least a fluid intake layer and an absorbent core. The fluid intake layer can be positioned between the topsheet layer and the absorbent core in the depth direction of the absorbent article. The fluid intake layer and the absorbent core can together define an aligned channel in the absorbent system. The aligned channel can create a void space in the depth direction of the absorbent article wherein at least a portion of the topsheet layer can be maintained in a spaced apart relationship from the bottom of the aligned channel. In various embodiments, the topsheet layer and/or the fluid intake layer can be treated with a surfactant.

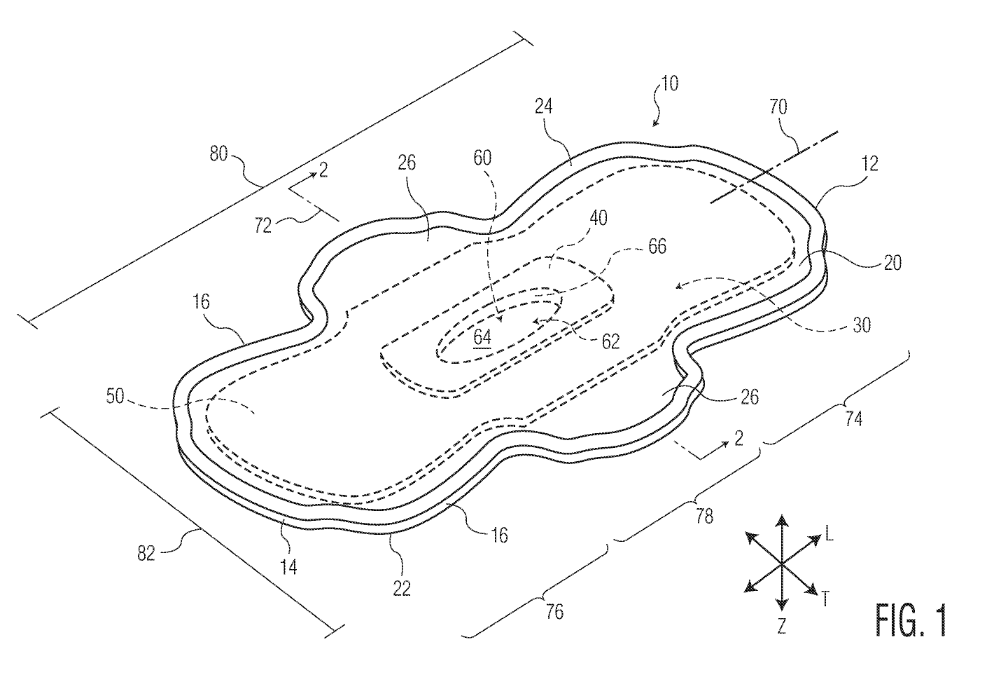

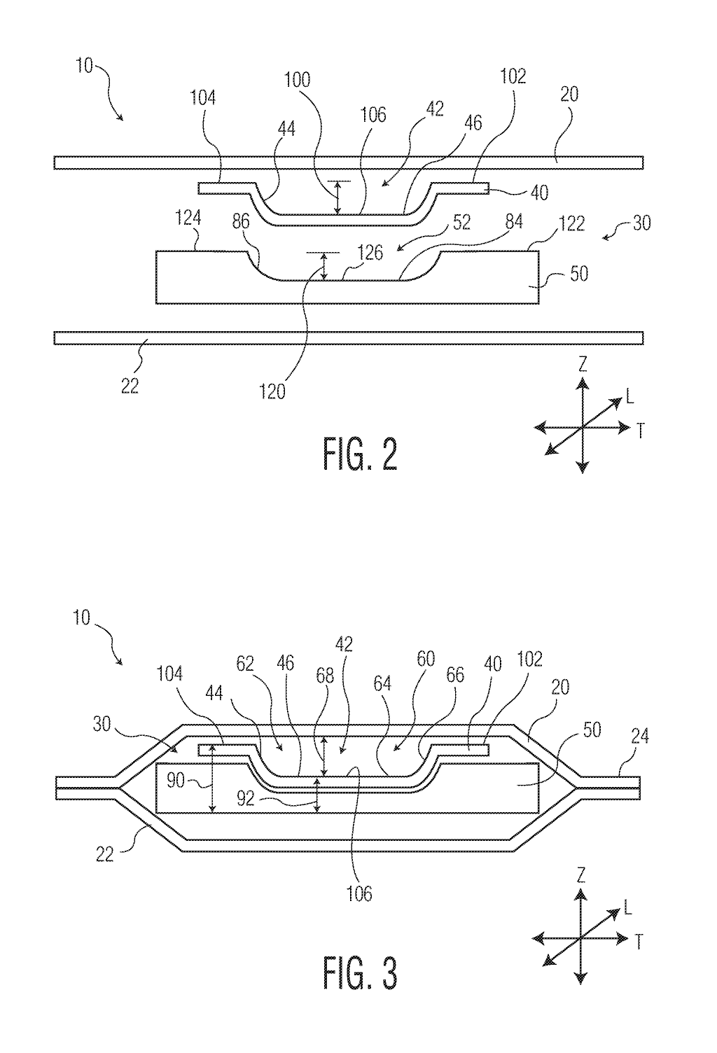

Referring to FIGS. 1-3, FIG. 1 provides an illustration of a perspective view of an exemplary absorbent article 10 in the form of a sanitary napkin with wings, FIG. 2 provides an illustration of an exploded cross-sectional view of the absorbent article 10 of FIG. 1 taken along line 2-2, and FIG. 3 provides an illustration of a cross-sectional view of the absorbent article 10 of FIG. 1 taken along line 2-2 wherein the fluid intake layer 40 is in a nested configuration with the absorbent core 50.

The absorbent article 10 can have a longitudinal direction (L), a transverse direction (T), and a depth direction (Z). The absorbent article 10 can have a first transverse direction end edge 12, a second transverse direction end edge 14 opposite the first transverse direction end edge 12, and a pair of opposing longitudinal direction side edges 16. The absorbent article 10 can have a wearer facing, liquid permeable topsheet layer 20 and a garment facing, liquid impermeable backsheet layer 22. An absorbent system 30 can be positioned between the topsheet layer 20 and the backsheet layer 22. The absorbent system 30 can include a fluid intake layer 40 and an absorbent core 50. The fluid intake layer 40 and the absorbent core 50 can together define an aligned channel 60 in the absorbent system 30. A portion of the absorbent system 30 can define a first absorbent system height 90 in the depth direction (Z) and a different portion of the absorbent system 30 can define a second absorbent system height 92 in the depth direction (Z). In various embodiments, the absorbent article 10 can take on various geometries but will generally have a pair of opposing longitudinal direction side edges 16 and a pair of opposing transverse direction end edges, 12 and 14.

The topsheet layer 20 and the backsheet layer 22 can both extend beyond the outermost peripheral edges of the absorbent system 30 and can be peripherally bonded together, either entirely or partially, using known bonding techniques to form a sealed peripheral region 24. For example, the topsheet layer 20 and the backsheet layer 22 can be bonded together by adhesive bonding, ultrasonic bonding, or any other suitable bonding method known in the art. In various embodiments, the topsheet layer 20 can further be bonded to the fluid intake layer 40 and/or the absorbent core 50. For example, in various embodiments, the topsheet layer 20 can be bonded to the fluid intake layer 40 and/or the absorbent core 50 in the area of the absorbent article 10 proximate to the aligned channel 60 without being bonded to the fluid intake layer 40 and/or absorbent core 50 within the aligned channel 60 thereby maintaining a spaced apart relationship with the bottom of the aligned channel 60. The bonding of the topsheet layer 20 to the fluid intake layer 40 and/or absorbent core 50 can occur via adhesive bonding, ultrasonic bonding, or any other suitable bonding method known in the art.

In various embodiments, such as illustrated in FIG. 1, the absorbent article 10 can have a pair of wings 26 extending outwardly, in the transverse direction (T), from the absorbent article 10. The wings 26 can drape over the edges of the wearer's undergarment so that the wings 26 are disposed between the edges of the wearer's undergarment and her thighs. The wings 26 can serve at least two purposes. First, the wings 26 can prevent soiling of the wearer's undergarment by forming a barrier along the edges of the undergarment. Second, the wings 26 can be provided with an attachment aid, such as, for example, a garment attachment adhesive or a hook, to keep the absorbent article 10 securely and properly positioned in the undergarment. The wings 26 can wrap around the crotch region of the wearer's undergarment to aid in securing the absorbent article 10 to the wearer's undergarment when in use. Each wing 26 can fold under the crotch region of the wearer's undergarment and the attachment aid can either form a secure attachment to the opposite wing 26 or directly to the surface of the wearer's undergarment. In various embodiments, the wings 26 can be an extension of materials forming the topsheet layer 20 and/or the backsheet layer 22, such that the wings 26 can be of a unitary construction with the absorbent article 10. In various embodiments, the wings 26 can be constructed of materials similar to the topsheet layer 20, the backsheet layer 22, the absorbent system 30, or combinations of these materials. In various embodiments, the wings 26 can be separate elements bonded to the main body of the absorbent article 10. It is to be understood that the wings 26 are optional and, in various embodiments, an absorbent article 10 can be configured without wings 26.

The absorbent article 10 defines a longitudinal direction (L), a transverse direction (T), a depth direction (Z), a longitudinal centerline 70, and a transverse centerline 72. The absorbent article 10 includes a front portion 74, a rear portion 76, and a central portion 78 extending between the front portion 74 and the rear portion 76. In general, the front portion 74 of the absorbent article 10 is adapted to be worn towards the front of the wearer, the central portion 78 is adapted to be worn proximate the wearer's crotch, and the rear portion 76 is adapted to be worn towards the rear of the wearer. The absorbent article 10 also defines an absorbent article length 80 in the longitudinal direction (L) and an absorbent article width 82 in the transverse direction (T). The front portion 74 of the absorbent article 10 is defined as the front third of the absorbent article length 80, the central portion 78 is defined as the center third of the absorbent article length 80, and the rear portion 76 is defined as the rear third of the absorbent article length 80. In various embodiments, the absorbent article 10 can have an absorbent article length 80, as measured at the longitudinal centerline 70, from about 200, 210, or 220 mm to about 240, 280, or 320 mm. In various embodiments, the absorbent article 10 can have an absorbent article width 82, as measured at the transverse centerline 72, from about 130, 135, or 140 mm to about 150, 155, or 160 mm for absorbent articles 10 with wings 26. In various embodiments, the absorbent article 10 can have an absorbent article width 82, as measured at the transverse centerline 72, from about 50, 60, or 65 mm to about 75, 80, or 90 mm for absorbent articles 10 without wings 26.

Each of these components of the absorbent article 10 will be described in more detail herein.

Topsheet Layer:

The topsheet layer 20 defines a wearer facing surface of the absorbent article 10 that may directly contact the body of the wearer and is liquid permeable to receive body exudates. The topsheet layer 20 is desirably provided for comfort and conformability and functions to direct body exudates away from the body of the wearer, through its own structure, and towards the absorbent system 30. The topsheet layer 20 desirably retains little to no liquid in its structure, so that it provides a relatively comfortable and non-irritating surface next to the skin of the wearer of the absorbent article 10.

The topsheet layer 20 can be a single layer of material, or alternatively, can be multiple layers that have been laminated together. The topsheet layer 20 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 20.

In various embodiments, the topsheet layer 20 can be constructed from various nonwoven webs such as meltblown webs, spunbond webs, hydroentangled spunlace webs, or through air bonded carded webs. Examples of suitable topsheet layer 20 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 20 can be a bonded carded web made of polypropylene and polyethylene such as that obtainable from Sandler Corporation, Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 20, each of which is hereby incorporated by reference thereto in its entirety. Additional topsheet layer 20 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 20 may contain a plurality of apertures formed therethrough to permit body exudates to pass more readily into the absorbent system 30. The apertures may be randomly or uniformly arranged throughout the topsheet layer 20. The size, shape, diameter, and number of apertures may be varied to suit an absorbent article's 10 particular needs.

In various embodiments, the topsheet layer 20 can have a basis weight ranging from about 5, 10, 15, 20 or 25 gsm to about 50, 100, 120, 125 or 150 gsm. For example, in an embodiment, a topsheet layer 20 can be constructed from a through air bonded carded web having a basis weight ranging from about 15 gsm to about 100 gsm. In another example, a topsheet layer 20 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics and others.

In various embodiments, the topsheet layer 20 can be at least partially hydrophilic. In various embodiments, the hydrophilicity of the topsheet layer 20 can be increased or created via treatment of the topsheet layer 20 with surfactants. In various embodiments, a portion of the topsheet layer 20 can be hydrophilic and a portion of the topsheet layer 20 can be hydrophobic. In various embodiments, the portions of the topsheet layer 20 which can be hydrophobic can be either an inherently hydrophobic material or can be a material treated with a hydrophobic coating.

In various embodiments, the topsheet layer 20 can be a multicomponent topsheet layer 20 such as by having two or more different nonwoven or film materials, with the different materials placed in separate locations in the transverse direction (T) of the absorbent article 10. For example, the topsheet layer 20 can be a two layer or multicomponent material having a central portion positioned along and straddling the longitudinal centerline 70 of the absorbent article 10, with lateral side portions flanking and bonded to each side edge of the central portion. The central portion can be constructed from a first material and the side portions can be constructed from a material which can be the same as or different from the material of the central portion. In such embodiments, the central portion may be at least partially hydrophilic and the side portions may be inherently hydrophobic or may be treated with a hydrophobic coating. Examples of constructions of multi-component topsheet layers 20 are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

In various embodiments, a central portion of a multicomponent topsheet layer 20 can be positioned symmetrically about the absorbent article 10 longitudinal centerline 70. Such central longitudinally directed central portion can be a spunbond or through air bonded carded web (“TABCW”) having a basis weight between about 15 and about 100 gsm. Previously described nonwoven, woven, and apertured film topsheet layer materials may also be used as the central portion of a multicomponent topsheet layer 20. In various embodiments, the central portion can be constructed from a TABCW material having a basis weight from about 20 to about 50 gsm such as is available from Xiamen Yanjan Industry, Beijing, DaYuan Nonwoven Fabrics, and others. Alternatively, apertured films, such as those available from such film suppliers as Texol, Italy and Tredegar, U.S.A. may be utilized. Different nonwoven, woven, or film sheet materials may be utilized as the side portions of the multicomponent topsheet layer 20. The selection of such topsheet layer 20 materials can vary based upon the overall desired attributes of the topsheet layer 20. For example, it may be desired to have a hydrophilic material in the central portion and hydrophobic-barrier type materials in the side portions to prevent leakage and increase a sense of dryness in the area of the side portions. Such side portions can be adhesively, thermally, ultrasonically, or otherwise bonded to the central portion along or adjacent the longitudinally directed side edges of the central portion. Traditional absorbent article construction adhesive may be used to bond the side portions to the central portion. Either of the central portion and/or the side portions may be treated with surfactants and/or skin-health benefit agents, as are well known in the art.

Such longitudinally directed side portions can be of a single or multi-layered construction. In various embodiments, the side portions can be a through air bonded carded web (“TABCW”). In various embodiments, the side portions can be adhesively or otherwise bonded laminates. In various embodiments, the side portions can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as a polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall topsheet layer 20 design, it may include opacifying agents, such as film pigments, that can help the film in masking stains along the absorbent article 10 side edges, thereby serving as a masking element. In such a fashion, the film layer can serve to limit visualization of a fluid insult stain along the absorbent article 10 side edges when viewed from above the topsheet layer 20. The film layer may also serve as a barrier layer to prevent rewet of the topsheet layer 20 as well as to prevent the flow of fluid off the side edges of the absorbent article 10. In various embodiments, the side portions can be laminates such as a spunbond-meltblown-meltblown-spunbond layer (“SMMS”) laminate, spunbond-film laminate, or alternatively, other nonwoven laminate combinations.

Absorbent System:

The absorbent system 30 can include a fluid intake layer 40 and an absorbent core 50. It is to be understood that the absorbent system 30 may include additional layers, which are in addition to the fluid intake layer 40 and the absorbent core 50, as are known in the art. The fluid intake layer 40 and the absorbent core 50 can define an aligned channel 60 in the absorbent system 30. The aligned channel 60 can create a void space 62 in the depth direction (Z) of the absorbent article 10. The aligned channel 60 has a bottom surface 64 and a perimeter side wall 66 and at least a portion of the topsheet layer 20 can be maintained in a spaced apart relationship from the bottom surface 64 of the aligned channel 60. The topsheet layer 20, the bottom surface 64 of the aligned channel 60, and perimeter side wall 66 of the aligned channel 60 define the boundaries of the void space 62. The aligned channel 60 can provide for improved handling of body exudates by allowing body exudates to pass through the topsheet layer 20 and into the void space 62 defined by the aligned channel 60 and the topsheet layer 20. The body exudates can then pass from the void space 62 defined by the aligned channel 60 and the topsheet layer 20 into the absorbent system 30. The aligned channel 60 minimizes the amount of body exudates which are in contact with the wearer's skin. Thus, it can be possible to minimize or prevent body exudates from leaking outwardly from the absorbent article 10 prior to the body exudates being ultimately absorbed into the absorbent system 30.

The aligned channel 60 can be formed by incorporating a channel into each of the fluid intake layer 40 and the absorbent core 50 and aligning the two channels together. The fluid intake layer channel 42 can be positioned in a nested configuration with the absorbent core channel 52 to form the aligned channel 60. In various embodiments in which the absorbent core 50 is formed of a single layer, the absorbent core channel 52 can be incorporated into the single layer of the absorbent core 50. In various embodiments in which the absorbent core 50 is formed of multiple layers of material, the absorbent core channel 52 can be incorporated into either only the body facing layer 54 of the absorbent core 50 or into both the body facing layer 54 and the garment facing layer 56 of the absorbent core 50.

The fluid intake layer channel 42 and the absorbent core channel 52 can be incorporated into their respective layer, fluid intake layer 40 or absorbent core 50, using various forming techniques. In various embodiments, the fluid intake layer channel 42 and the absorbent core channel 52 can be incorporated into their respective layer, fluid intake layer 40 or absorbent core 50, during the formation of the layer of material such as by utilizing a shaped forming drum. In various embodiments, the fluid intake layer channel 42 and the absorbent core channel 52 can be incorporated into their respective layer, fluid intake layer 40 or absorbent core 50, utilizing an embossing roll. In various embodiments of a manufacturing process, the fluid intake layer channel 42 and the absorbent core channel 52 can be incorporated into their respective layer, fluid intake layer 40 or absorbent layer 50, utilizing the same technique. In various embodiments of a manufacturing process, the fluid intake layer channel 42 and the absorbent core channel 52 can be incorporated into their respective layer, fluid intake layer 40 or absorbent core 50, utilizing differing techniques. In various embodiments, the fluid intake layer channel 42 and the absorbent core channel 52 can be incorporated into their respective layer, fluid intake layer 40 or absorbent core 50, at the same time in a manufacturing process. In various embodiments, the fluid intake layer 40 can be superimposed on the absorbent core 50 and the fluid intake layer channel 42 and the absorbent core channel 52 can be incorporated into the superimposed fluid intake layer 40 and absorbent core 50 at the same time. In various embodiments, the fluid intake layer channel 42 and the absorbent core channel 52 can be incorporated into their respective layer, fluid intake layer 40 or absorbent core 50, at different times in a manufacturing process. In various embodiments, the fluid intake layer channel 42 and the absorbent core channel 52 can be incorporated into their respective layer, fluid intake layer 40 or absorbent core 50, separately followed by nesting the fluid intake layer channel 42 into the absorbent core channel 52.

The fluid intake layer channel 42 can provide a three-dimensional structure to the fluid intake layer 40 and the absorbent core channel 52 can provide a three-dimensional structure to the absorbent core 50. Each of the channels, 42 and 52, can have a bottom surface and perimeter side walls. The fluid intake layer channel 42 can have a fluid intake layer channel bottom surface 46 and a fluid intake layer channel perimeter side wall 44. The absorbent core channel 52 can have an absorbent core channel bottom surface 84 and an absorbent core channel perimeter side wall 86. The fluid intake layer channel 42 can be placed into a nested configuration with the absorbent core channel 52. In the nested configuration, the fluid intake layer channel 42 can contact the absorbent core channel 52 and such contact can provide improved transfer of body exudates. The contact between the fluid intake layer channel 42 and the absorbent core channel 52 can also protect the three-dimensional structure of the absorbent core 50 as well as the absorbent core channel 52. In various embodiments, the absorbent core 50 can be formed of a moisture sensitive material and the fluid intake layer 40 can be formed of a moisture insensitive material. In such embodiments, the moisture sensitive material forming the absorbent core channel 52 may expand upon contact with fluid and lose the shape of the absorbent core channel 52. In such embodiments, the moisture insensitive material forming the fluid intake layer channel 42 may refrain from expansion upon contact with fluid and, therefore, maintain the shape of the fluid intake layer channel 42. As the fluid intake layer channel 42 is in a nested configuration with the absorbent core channel 52, the lack of expansion and the lack of a loss of shape of the fluid intake layer channel 42 can enable the absorbent core channel 52 to maintain its shape upon contact with a fluid.

The fluid intake layer channel 40, the absorbent core channel 50, and the aligned channel 60 can each be any suitable shape, such as ovular, circular, rectangular, square, triangular, etc. The shape of the aligned channel 60 can be provided by the shapes of the fluid intake layer channel 40 and the absorbent core channel 50. In various embodiments, each of the fluid intake layer channel 42, the absorbent core channel 52 and the aligned channel 60 can have the same shape. In various embodiments, each of the fluid intake layer channel 42, the absorbent core channel 52, and the aligned channel 60 can have a different shape while the absorbent core channel 52 has a shape that the can allow for a nested configuration with the fluid intake layer channel 42 shape. In various embodiments, the aligned channel 60 in the absorbent article 10 can be elongate and can be oriented in the longitudinal direction (L) of the absorbent article 10. In such embodiments, when the aligned channel 60 is elongate and oriented in the longitudinal direction (L) of the absorbent article 10, the body exudates may tend to be absorbed and spread in the longitudinal direction (L) of the absorbent article 10 rather than in the transverse direction (T) of the absorbent article 10. Accordingly, it can be possible to minimize or prevent the body exudates from heavily spreading in the transverse direction (T) of the absorbent article 10 and thereby leaking out of the absorbent article 10.

Each of the channels, 42 and 52, can include a depth, 100 and 120, respectively, in the depth direction (Z) of the fluid intake layer 40 and the absorbent core 50, respectively. For purposes herein, the depth 100 of the fluid intake layer channel 42 is defined in the depth direction (Z) between the body facing surface 102 of the fluid intake layer 40 at a first position 104 that does not include the fluid intake layer channel 42 and the body facing surface 102 of the fluid intake layer 40 at a second position 106 forming the lowest point of the fluid intake layer channel 42. In various embodiments, the depth 100 of the fluid intake layer channel 42 can be from about 0.5, 1.0, or 2.0 mm to about 5.0, 10.0, or 20.0 mm. It is contemplated that the depth 100 of the fluid intake layer channel 42 can be outside these described ranges and still be within the scope of the present disclosure. For purposes herein, the depth 120 of the absorbent core channel 52 is defined in the depth direction (Z) between the body facing surface 122 of the absorbent core 50 at a first position 124 that does not include the absorbent core channel 52 and the body facing surface 122 of the absorbent core 50 at a second position 126 forming the lowest point of the absorbent core channel 52. In various embodiments, the depth 120 of the absorbent core channel 52 can be from about 0.5, 1.0, or 2.0 mm to about 5.0, 10.0, or 20.0 mm. It is contemplated that the depth 120 of the absorbent core channel 52 can be outside these described ranges and still be within the scope of the present disclosure. In various embodiments, the depth 120 of the absorbent core channel 52 is deep enough to allow for the nesting configuration wherein the fluid intake layer channel 42 is placed into a nested configuration with the absorbent core channel 52. The nested configuration of the fluid intake layer channel 42 in the absorbent core channel 52 defines the aligned channel 60 of the absorbent article 10.

Referring to FIG. 3 which is an illustration of a cross-sectional view of the absorbent article of FIG. 1 taken along line 2-2, the fluid intake layer channel 42 is illustrated in a nested configuration with the absorbent core channel 52 thereby forming the aligned channel 60 of the absorbent article 10. The aligned channel 60 can have a bottom surface 64 which can be the same as the fluid intake layer channel bottom surface 46. The aligned channel 60 can have a perimeter side wall 66 which is the same as the perimeter side wall 44 of the fluid intake layer channel perimeter side wall 44.

The topsheet layer 20 can be in a spaced apart relationship from the bottom surface 64 of the aligned channel 60 such that a spatial gap exists between the topsheet layer 20 and the bottom surface 64 of the aligned channel 60. In remaining spaced apart from the bottom surface 64 of the aligned channel 60, the topsheet layer 20 does not contact the bottom surface 64 of the aligned channel 60 and the spatial gap creates a void space 62 between the topsheet layer 20 and the bottom surface 64 of the aligned channel 60. In other words, the spatial gap is the gap between the layers of the absorbent article 10, in the depth direction (Z) of the absorbent article 10, in which a void space 62 is present, wherein the void space 62 is not the result of normal interstitial spaces between immediately adjacent layers and is not the result where construction adhesive between immediately adjacent layers are absent. The aligned channel 60 can have an aligned channel height 68 which is defined in the depth direction (Z) as the measured distance between the topsheet layer 20 and the body facing surface 102 of the fluid intake layer 40 at a position 106 forming the lowest point of the aligned channel 60. In various embodiments, the aligned channel height 68 can be the same as the fluid intake layer channel depth 100. In various embodiments, the aligned channel height 68 can be from about 0.5, 1.0, or 2.0 mm to about 5.0, 10.0, or 20.0 mm. It is contemplated that the aligned channel height 68 can be outside these described ranges and still be within the scope of the present disclosure.

The aligned channel 60 can be located at various positions along the longitudinal direction (L) and the transverse direction (T) of the absorbent article 10 depending upon the primary location of body exudate intake or the purpose for which the absorbent article 10 is being used. For example, in various embodiments, the aligned channel 60 can be positioned so that it is in substantial alignment with the longitudinal centerline 70 and the transverse centerline 72 of the absorbent article 10. It should be understood that the longitudinal centerline 70 is disposed at a location that is equidistant from the longitudinal direction side edges 16 and runs the length of the absorbent article 10 in the longitudinal direction (L) while the transverse centerline 72 is disposed at a location that is equidistant from the first transverse direction end edge 12 and the second transverse direction end edge 14 and runs the width of the absorbent article 10 in the transverse direction (T). The aligned channel 60 can be centrally disposed so that it can be positioned below the main point of body exudate discharge and so that it can act as the primary body exudate receiving area for the absorbent article 10.

However, centralized positioning of the aligned channel 60 is not required, and in various embodiments, depending on the primary location where body exudate intake might occur, the aligned channel 60 of the absorbent article 10 may be substantially aligned with the longitudinal centerline 70 only. Thus, in various embodiments, the aligned channel 60 of the absorbent article 10 may be shifted in the longitudinal direction towards either of the transverse direction end edges, 12 or 14, of the absorbent article 10, so that the aligned channel 60 of the absorbent article 10 is not in substantial alignment with the transverse centerline 72.

The aligned channel 60 of the absorbent article 10 can have a longitudinal length from about 15, 20, 30, or 50 mm to about 60, 75, 100, or 150 mm and can have a transverse width from about 10, 15, 20, or 30 mm to about 40, 50, 60, or 80 mm. The aligned channel 60 in the absorbent article 10 can have a longitudinal length that is from about 15, 20, or 25% to about 70, 75, or 80% of the overall longitudinal length of the absorbent article 10 in the longitudinal direction (L). The aligned channel 60 in the absorbent article can have a transverse width that can be from about 20, 25, or 30% to about 70, 75, or 80% of the overall transverse width of the absorbent article 10 in the transverse direction (T).

In various embodiments, the aligned channel 60 can be fully positioned within the central portion 78 of the absorbent article 10. In various embodiments, the aligned channel 60 can have a portion positioned within the front portion 74 of the absorbent article 10 and a portion positioned within the central portion 78 of the absorbent article 10. In various embodiments, the aligned channel 60 can have a portion positioned with the rear portion 76 of the absorbent article 10 and a portion positioned within the central portion 78 of the absorbent article 10. In various embodiments, the aligned channel 60 can have a portion positioned in each of the front portion 74, central portion 78, and rear portion 76 of the absorbent article 10. In various embodiments, the aligned channel 60 can be fully positioned within the rear portion 76 of the absorbent article 10 such as may be desired in an absorbent article 10 for overnight usage.

While the Figures illustrate a single aligned channel 60 incorporated into the absorbent article 10, it is to be understood that the absorbent article 10 can have more than one aligned channel 60. In various embodiments, the plurality of aligned channels 60 can be in a pattern that can form a plurality of rows in the transverse direction (T) and/or a plurality of columns in the longitudinal direction (L) of the absorbent article 10. In various embodiments, the plurality of aligned channels 60 can be uniformly distributed through the longitudinal length 80 and/or transverse width 82 of the absorbent article 10. In various embodiments, the plurality of aligned channels 60 can be non-uniformly distributed throughout the longitudinal length 80 and/or transverse width 82 of the absorbent article 10.

Incorporating channels, 42 and 52, into the fluid intake layer 40 and absorbent core 50, respectively, can result in an absorbent system 30 which can have at least two portions with differing heights. In various embodiments, the region of the absorbent system 30 wherein the aligned channel 60 is positioned can have a first height 92 and the region of the absorbent system 30 without the aligned channel 60 can have a second height 90. The second height 90 of the absorbent system 30 can be greater than the first height 92 of the absorbent system 30.

Incorporating channels, 42 and 52, into the fluid intake layer 40 and the absorbent core 50, respectively, can result in an absorbent system 30 which can have at least two portions with differing densities. In various embodiments, the region of the absorbent system 30 wherein the aligned channel 60 is positioned can have a first density and the region of the absorbent system 30 without the aligned channel 60 can have a second density. In various embodiments, the first density can be greater than the second density. Alternatively, in various embodiments, the first density can be less than the second density.

In various embodiments, the absorbent article 10 can have at least one embossment positioned in the front portion 74, central portion 78, rear portion 76, and combinations thereof. The embossment can be incorporated into the absorbent article 10 utilizing known techniques such as, for example, an embossing roll. The additional embossment(s), if present, can transfer compressive forces applied by the legs of the wearer during wear and usage of the absorbent article 10. Such transfer of compressive forces can further allow the absorbent article 10 to conform to the wearer's body during wear and usage of the absorbent article 10. In various embodiments, an embossment can include a portion of the topsheet layer 20 embossed into a portion of the fluid intake layer 40. In various embodiments, an embossment can include a portion of the topsheet layer 20 embossed into a portion of the fluid intake layer 40 and a portion of the absorbent core 50. In various embodiments, an embossment can include a portion of the topsheet layer 20 embossed into a portion of the absorbent core 50, such as, for example, can occur in portions of the absorbent article 10 where the fluid intake layer 40 is not located.

Fluid Intake Layer:

In various embodiments, the absorbent article 10 can include a liquid permeable fluid intake layer 40 positioned between the topsheet layer 20 and the absorbent core 50. Such a fluid intake layer 40 can be made of a material that can be capable of rapidly transferring, in the depth direction (Z), body exudates that are delivered to the topsheet layer 20. The fluid intake layer 40 can generally have any shape and/or size desired. In an embodiment, the fluid intake layer 40 can have a curved rectangular shape, with a length equal to or less than the overall length of the absorbent article 10, and a width equal to or less than the width of the absorbent article 10. For example, the fluid intake layer 40 can have a longitudinal length of between about 20, 40, or 60 mm to about 150, 150, 175, 200, or 300 mm and a transverse width of between about 10, 15 or 20 mm to about 60, 80 or 100 mm may be utilized. The fluid intake layer 40 can have a height in the depth direction (Z) from about 0.5 mm to about 3 mm. The fluid intake layer 40 can have a basis weight from about 10, 25, or 100 gsm to about 200, 250, or 300 gsm. In various embodiments, the fluid intake layer 40 can have substantially the same longitudinal length and transverse width dimensions as the absorbent article 10. In various embodiments, the fluid intake layer 40 can have a longitudinal length and a transverse width dimension which are smaller than the longitudinal length 80 and transverse width 82 of the absorbent article 10. In various embodiments, the fluid intake layer 40 can have a longitudinal length and a transverse width dimension sufficient to form a fluid intake layer channel 42 which can be placed into a nested configuration with an absorbent core channel 52 to protect the three-dimension structure of the absorbent core channel 52 when in the nested configuration. In various embodiments, the absorbent article 10 can have more than one aligned channel 60 and can have a single fluid intake layer 40 forming a portion of each of the aligned channels 60. In various embodiments, the absorbent article 10 can have more than one aligned channel 60 and each of the aligned channels 60 can have its own fluid intake layer 40 forming a portion of the aligned channel 60.

Any of a variety of different materials can be capable of being used for the fluid intake layer 40 to accomplish the above-mentioned functions. The material may be synthetic, cellulosic, or a combination of synthetic and cellulosic materials. The fluid intake layer 40 can be constructed from any woven or nonwoven material. For example, the fluid intake layer 40 can be constructed as an airlaid, spunbond, tissue, meltblown, spunbond-meltblown-spunbond, or TABCW material. For example, airlaid cellulosic tissues may be suitable for use in the fluid intake layer 40. The airlaid cellulosic tissue may have a basis weight ranging from about 10 or 100 gsm to about 250 or 300 gsm. The airlaid cellulosic tissue can be formed from hardwood and/or softwood fibers. An airlaid cellulosic tissue can have a fine pore structure and can provide an excellent wicking capacity, especially for menses. In various embodiments, the fluid intake layer 40 can be at least partially hydrophilic. In various embodiments, the hydrophilicity of the fluid intake layer 40 can be increased or created via treatment of the fluid intake layer 40 with surfactants.

Additionally, to further enhance the ability of the absorbent article 10 to transfer body exudates in the depth direction (Z) from the topsheet layer 20 toward any lower layers in the absorbent article 10 as well as to enhance the ability of the fluid intake layer 40 to conform to the wearer's body based on its ability to bend, the fluid intake layer 40 can have a fluid intake layer channel 42 in the fluid intake layer 40 which can be any suitable shape, such as ovular, circular, rectangular, square, triangular, etc. In various embodiments, the fluid intake layer channel 42 in the fluid intake layer 40 can be elongate and can be oriented in the longitudinal direction (L) of the absorbent article 10. The fluid intake layer channel 42 in the fluid intake layer 40 can be bounded by a perimeter side wall 44.

The fluid intake layer channel 42 can be located at various positions along the longitudinal and transverse directions of the fluid intake layer 40 depending upon the primary location of body exudate intake or the purpose for which the absorbent article 10 is being used. For example, in various embodiments, the fluid intake layer 40 and the fluid intake layer channel 42 can be positioned so that they are in substantial alignment with the longitudinal centerline 70 and the transverse centerline 72 of the absorbent article 10. This allows the fluid intake layer channel 42 to be centrally disposed so that it can be positioned below the main point of body exudate discharge and so that it can act as the primary body exudate receiving area for the absorbent article 10.

However, centralized positioning of the fluid intake layer 40 and the fluid intake layer channel 42 is not required, and in various embodiments, depending on the primary location where body exudate intake might occur, the fluid intake layer 40 and the fluid intake layer channel 42 may be substantially aligned with the longitudinal centerline 70 only. Thus, in various embodiments, the fluid intake layer 40 and the fluid intake layer channel 42 may be shifted in the longitudinal direction (L) towards either transverse direction end edge, 12 or 14, of the absorbent article 10, so that the fluid intake layer channel 42 is not in substantial alignment with the transverse centerline 72.

The fluid intake layer channel 42 can have a longitudinal length from about 15, 20, 30, or 50 mm to about 60, 75, 100, or 150 mm and can have a transverse width from about 10, 15, 20, or 30 mm to about 40, 60, or 80 mm. The fluid intake layer channel 42 can have a length that is from about 15, 20, or 25% to about 70, 75, or 80% of the overall longitudinal length of the fluid intake layer 40 in the longitudinal direction (L). The fluid intake layer channel 42 can have a width that can be from about 20, 25, or 30% to about 70, 75, or 80% of the overall width of the fluid intake layer 40 in the transverse direction (T).

In various embodiments, the fluid intake layer channel 42 can be fully positioned within the central portion 78 of the absorbent article 10. In various embodiments, the fluid intake layer channel 42 can have a portion positioned within the front portion 74 of the absorbent article 10 and a portion positioned within the central portion 78 of the absorbent article 10. In various embodiments, the fluid intake layer channel 42 can have a portion positioned with the rear portion 76 of the absorbent article 10 and a portion positioned within the central portion 78 of the absorbent article 10. In various embodiments, the fluid intake layer channel 42 can have a portion positioned in each of the front portion 74, central portion 78, and rear portion 76 of the absorbent article 10. In various embodiments, the fluid intake layer channel 42 can be fully positioned within the rear portion 76 of the absorbent article 10 such as may be desired in an absorbent article 10 for overnight usage.

Absorbent Core:

An absorbent core 50 can be positioned between the topsheet layer 20 and the backsheet layer 22. The absorbent core 50 can be positioned beneath the fluid intake layer 40 in the depth direction of the absorbent article 10. The absorbent core 50 can generally be any single layer structure or combination of layer components, which can demonstrate some level of compressibility, conformability, be non-irritating to a wearer's skin, and capable of absorbing and retaining liquids and other body exudates. Additionally, the absorbent core 50 can provide additional capacity to absorb and retain body exudates such as menses. In various embodiments, the absorbent core 50 can be formed from a variety of different materials and can contain any number of desired layers. For example, the absorbent core 50 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers.

In various embodiments, if desired, the absorbent core 50 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 50 in any amount as desired.

Regardless of the combination of absorbent materials used in the absorbent core 50, the absorbent materials can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

The shape of the absorbent core 50 can vary as desired and can comprise any one of various shapes including, but not limited to, triangular, rectangular, dog-bone and elliptical shapes. In various embodiments, the absorbent core 50 can have a shape that generally corresponds with the overall shape of the absorbent article 10. The dimensions of the absorbent core 50 can be substantially similar to those of the absorbent article 10, however, it will be appreciated that the dimensions of the absorbent core 50 while similar, will often be less than those of the overall absorbent article 10, in order to be adequately contained therein.

By way of example, suitable materials and/or structures for the absorbent core 50 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al., each of which is hereby incorporated by reference thereto in its entirety.

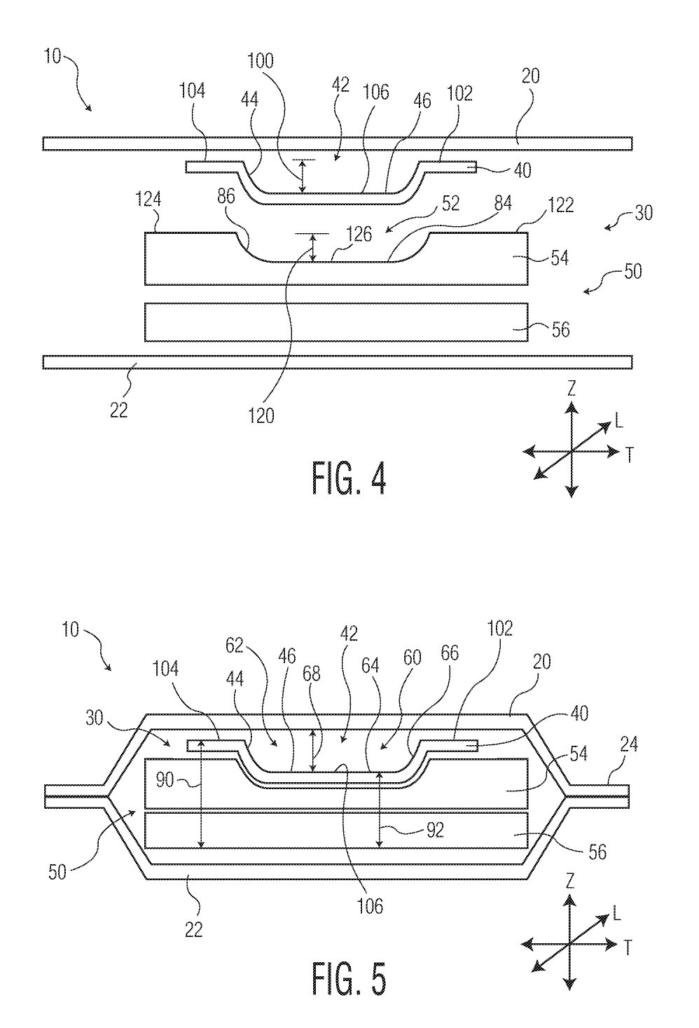

As described above, in various embodiments, an absorbent core 50 can be a single layer structure and can include, for example, a matrix of cellulosic fluff and superabsorbent material. In various embodiments, such as illustrated in FIGS. 4 and 5 herein, an absorbent core 50 can have at least two layers of material, such as, for example, a body facing layer 54 and a garment facing layer 56. In various embodiments, the two layers, 54 and 56, can be identical to each other. In various embodiments, the two layers, 54 and 56, can be different from each other. In such embodiments, the two layers, 54 and 56, can provide the absorbent article 10 with different absorption properties as deemed suitable. In various embodiments, the body facing layer 54 of the absorbent core 50 may be constructed of an airlaid material and the garment facing layer 56 of the absorbent core 50 may be constructed of a superabsorbent polymer-containing compressed sheet. In such embodiments, the airlaid material can have a basis weight from about 40 to about 200 gsm and the superabsorbent polymer-containing compressed sheet can be a cellulosic fluff based material that can be a combination of cellulosic pulp and SAP enclosed with a tissue carrier and having a basis weight from about 40 to about 400 gsm.

Additionally, to further enhance the ability of the absorbent article 10 to transfer body exudates in the depth direction (Z) from the topsheet layer 20 toward any lower layers in the absorbent article 10 as well as to enhance the ability of the absorbent core 50 to conform to the wearer's body based on its ability to bend, the absorbent core 50 can have an absorbent core channel 52 in the absorbent core 50 which can be any suitable shape, such as ovular, circular, rectangular, square, triangular, etc. In various embodiments in which the absorbent core 50 comprises a single layer of material, the absorbent core channel 52 can exist in the single layer composing the absorbent core 50. In various embodiments in which the absorbent core 50 is formed of two layers, such as a body facing layer 54 and a garment facing layer 56, the absorbent core 50 can have an absorbent core channel 52 in the body facing layer 54 and in the garment facing layer 56. In various embodiments, the absorbent core channel 52 in the absorbent core 50 can be elongate and can be oriented in the longitudinal direction (L) of the absorbent article 10.

The absorbent core channel 52 can be located at various positions along the longitudinal and transverse directions of the absorbent core 50 depending upon the primary location of body exudate intake or the purpose for which the absorbent article 10 is being used. For example, in various embodiments, the absorbent core channel 52 can be positioned so that it is in substantial alignment with the longitudinal centerline 70 and the transverse centerline 72 of the absorbent article 10. This allows the absorbent core channel 52 to be centrally disposed so that it can be positioned below the main point of body exudate discharge and so that it can act as the primary body exudate receiving area for the absorbent article 10.

However, centralized positioning of the absorbent core channel 52 is not required, and in various embodiments, depending on the primary location where body exudate intake might occur, the absorbent core channel 52 may be substantially aligned with the longitudinal centerline 70 only. Thus, in various embodiments, the absorbent core channel 52 may be shifted in the longitudinal direction (L) towards either transverse direction end edge, 12 or 14, of the absorbent article 10, so that the absorbent core channel 52 is not in substantial alignment with the transverse centerline 72.

The absorbent core channel 52 can have a longitudinal length from about 15, 20, 30 or 50 mm to about 60, 75, 100 or 150 mm and can have a transverse width from about 10, 15, 20 or 30 mm to about 40, 60 or 80 mm. The absorbent core channel 52 can have a length that is from about 15, 20 or 25% to about 70, 75, or 80% of the overall longitudinal length of the absorbent core 50 in the longitudinal direction (L). The absorbent core channel 52 can have a width that can be from about 20, 25 or 30% to about 70, 75 or 80% of the overall width of the absorbent core 50 in the transverse direction (T). The absorbent core channel 52 can serve to funnel and direct body exudates from the topsheet layer 20 and towards lower layers of the absorbent article 10 in the depth direction (Z). The absorbent core channel 52 can also form a cup or well-like structure for holding body exudates and preventing its leakage away from a central region of the absorbent article 10 and towards the edges of the absorbent article 10.

In various embodiments, the absorbent core channel 52 can be fully positioned within the central portion 78 of the absorbent article 10. In various embodiments, the absorbent core channel 52 can have a portion positioned within the front portion 74 of the absorbent article 10 and a portion positioned within the central portion 78 of the absorbent article 10. In various embodiments, the absorbent core channel 52 can have a portion positioned with the rear portion 76 of the absorbent article 10 and a portion positioned within the central portion 78 of the absorbent article 10. In various embodiments, the absorbent core channel 52 can have a portion positioned in each of the front portion 74, central portion 78, and rear portion 76 of the absorbent article 10. In various embodiments, the absorbent core channel 52 can be fully positioned within the rear portion 76 of the absorbent article 10 such as may be desired in an absorbent article 10 for overnight usage.

In various embodiments, the absorbent core channel 52 can align with the fluid intake layer channel 42. In such embodiments, the absorbent core channel 52 can have the same overall shape and dimensions as the fluid intake layer channel 42. In such embodiments, the absorbent core channel 52 and the fluid intake layer channel 42 can be positioned in the absorbent article 10 having the same spatial relationship with the longitudinal centerline 70 and the transverse centerline 72 of the absorbent article 10.

Backsheet Layer:

The backsheet layer 22 is generally liquid impermeable and is the portion of the absorbent article 10 which faces the garment of the wearer. The backsheet layer 22 can permit the passage of air or vapor out of the absorbent article 10 while still blocking the passage of liquids. Any liquid impermeable material may generally be utilized to form the backsheet layer 22. The backsheet layer 22 can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the backsheet layer 22 can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a backsheet layer 22 can be a polyethylene film such as that obtainable from Pliant Corporation, Schaumburg, Ill., USA. Another example can include calcium carbonate-filled polypropylene film. In still another embodiment, the backsheet layer 22 can be a hydrophobic nonwoven material with water barrier properties such as a nonwoven laminate, an example of which can be a spunbond, meltblown, meltblown, spunbond, four-layered laminate. The backsheet layer 22 can, therefore, be of a single or multiple layer construction, such as of multiple film layers or laminates of film and nonwoven fibrous layers. Suitable backsheet layers 22 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

Wings:

The wings 26 can be constructed from materials described above with respect to the topsheet layer 20 and the backsheet layer 22. In various embodiments, the wings 26 can comprise an extension of a layer of material within the topsheet layer 20 and/or the backsheet layer 22. By way of example, the wings 26 can be formed by an extension of the topsheet layer 20 and backsheet layer 22 that are then bonded together along sealed peripheral region 24. Such wings 26 can be integrally formed with the main portion of the absorbent article 10. Alternatively, the wings 26 can be formed independently and separately attached to an intermediate section of the absorbent article 10. Wings 26 that are made independent of the other components of the absorbent article 10 can be bonded to a portion of the topsheet layer 20 and/or backsheet layer 22. Examples of processes for manufacturing absorbent articles 10 and wings 26 include, but are not limited to, those described in U.S. Pat. No. 4,059,114 to Richards, U.S. Pat. No. 4,862,574 to Hassim, et al., U.S. Pat. No. 5,342,647 to Heindel, et al., U.S. Pat. No. 7,070,672 to Alcantara, et al., U.S. Publication No., 2004/0040650 to Venturino, et al., and international publication WO1997/040804 to Emenaker, et al., each of which are hereby incorporated by reference thereto in its entirety.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent article characterized by comprising:
a. a longitudinal direction, a transverse direction, and a depth direction;
b. a topsheet layer and a backsheet layer; and
c. an absorbent system positioned between the topsheet layer and the backsheet layer, the absorbent system comprising:
i. a fluid intake layer positioned below the topsheet layer in the depth direction of the absorbent article, wherein the fluid intake layer comprises a fluid intake layer channel;
ii. an absorbent core positioned below the fluid intake layer in the depth direction of the absorbent article, wherein the absorbent core comprises a fluid intake layer;
wherein the fluid intake layer channel is in a nested configuration with the absorbent core channel to form an aligned channel of the absorbent article, the aligned channel comprising an aligned channel bottom surface and an aligned channel perimeter side wall; and
wherein the topsheet layer is in a spaced apart relationship from the aligned channel bottom surface resulting in a spatial gap between the topsheet layer and the bottom surface of the aligned channel.

2. The absorbent article of claim 1 wherein the aligned channel is elongate.

3. The absorbent article of claim 1 wherein the aligned channel is oriented in the longitudinal direction of the absorbent article.

4. The absorbent article of claim 1 wherein the aligned channel comprises a shape wherein the shape is one of ovular, circular, rectangular, square or triangular.

5. The absorbent article of claim 1 wherein a first portion of the absorbent system has a first height and a second portion of the absorbent system has a second height and the first height is different from the second height.

6. The absorbent article of claim 1 wherein a first portion of the absorbent system has a first density and a second portion of the absorbent system has a second density and the first density is different from the second density.

7. The absorbent article of claim 6 wherein the first density is greater than the second density.

8. The absorbent article of claim 6 wherein the first density is less than the second density.

9. The absorbent article of claim 1 wherein the topsheet layer comprises a surfactant.

10. The absorbent article of claim 1 wherein the fluid intake layer comprises a surfactant.

11. The absorbent article of claim 1 wherein the absorbent core comprises at least two separate layers.

12. The absorbent article of claim 1 wherein the aligned channel has a longitudinal length from about 15 mm to about 150 mm.

13. The absorbent article of claim 1 wherein the aligned channel has a transverse width from about 10 mm to about 90 mm.

14. The absorbent article of claim 1 wherein the height of the spatial gap is from about 1 mm to about 8 mm.

15. The absorbent article of claim 1 wherein the fluid intake layer is formed of a moisture insensitive material and the absorbent core is formed of a moisture sensitive material.
